(19) United States
(12) Patent Application Publication
(10) Pub. No.: US 2014/0358094 A1
(43) Pub. Date: Dec. 4, 2014

(54) COMPOSITION, THE NANO-EMULSION AND TRANSDERMAL PATCH, METHODS OF PREPARATION AND USE THEREOF FOR TREATING TRAUMATIC INJURIES

(71) Applicant: Nano and Advanced Materials Institute Limited, Hong Kong (HK)

(72) Inventors: Ka Wing LAM, Hong Kong (HK); Chun Hay KO, Hong Kong (HK); Wing Sum SIU, Hong Kong (HK); Clara Bik San LAU, Hong Kong (HK); Tai Wai David LAU, Hong Kong (HK); Kam Ming KO, Hong Kong (HK); Ka Ming NG, Hong Kong (HK); Ping Chung LEUNG, Hong Kong (HK)

(73) Assignee: Nano and Advanced Materials Institute Limited, Hong Kong (HK)

(21) Appl. No.: 14/290,962

(22) Filed: May 29, 2014

Related U.S. Application Data
(60) Provisional application No. 61/855,966, filed on May 29, 2013.

(30) Foreign Application Priority Data
May 29, 2014 (CN) 201410232730.4

(51) Int. Cl. A61K 9/00; A61K 36/286; A61K 36/185; A61K 36/708; A61K 9/70; A61K 36/258

(52) U.S. Cl. CPC A61K 9/0014; A61K 9/7084; A61K 36/258; A61K 36/185; A61K 36/708; A61K 36/286
USPC 604/290; 424/728; 604/307

(57) ABSTRACT

The present invention is a herbal composition and method of preparing thereof. The herbal composition comprising extracts of Carthami Flos (CF), Dipsaci Radix (DR), Notoginseng Rhizoma (NR) and Rhei Rhizoma (RR). The present invention also relates to an oil-in-water or water-in-oil type nano-emulsion and method of preparing thereof. The nano-emulsion comprises an aqueous phase, an organic phase and a surfactant, wherein said aqueous phase comprises hydrophilic extract of herbal materials and said organic phase comprises hydrophobic extract of said herbal materials. The present invention also relates to a transdermal patch comprises said nano-emulsion. The herbal composition of the present invention is useful in treating traumatic injuries, in particular bone injuries. The present nano-emulsion and patch thereof can promote skin absorption of active ingredients in herb, thereby increasing the herb's bioavailability and efficacy in therapy.

### FIELD OF INVENTION

The present invention relates to a herbal composition for treating bone injuries, a nano-emulsion comprising said herbal composition and a transdermal patch comprising said nano-emulsion. The present invention also relates to method of preparing and use of the herbal composition, the nano-emulsion and the transdermal patch.

### BACKGROUND OF INVENTION

Traumatic musculoskeletal injury or fracture is one of the common observations in orthopaedic clinics. The world incidence of adult fractures is estimated to be around 9.0-22.8 per 1000 people per year. Considering that fractures are commonly observed in elderly, the number of fracture cases is expected to increase steadily in the future due to the aging population. Patients with bone fracture require long hospitalization before they can be discharged. The median length of stay after hip fracture fixation is 5 days, with highest 33 days in New York from 1997 to 1998; the mean length of stays of overall fracture in men and women are 13.3 and 19.6 days, respectively and the total days of hospitalization were over one million in Switzerland in 1992. Fracture also reduces the social productivity and increases the health services utilization and social-economical burden.

Although reduction and fixation are effectively managed by surgeons nowadays, post-operation healing process is seldom considered by hospital and clinical workers. After reduction of fractures, bone is immobilized by casting or fixators and then healing relies mostly on self-recovery. Patients are usually let unattended except for pain and inflammation control during hospitalization. In spite of many scientific researches in finding a way to promote fracture healing, including the use of biomaterial scaffolds, growth factors, bone morphogenetic proteins and biophysical stimulations, these interventions have not been well accepted for routine clinical applications.

Facilitation of fracture healing is one of the key areas in Traditional Chinese Medicine (TCM). Almost all the TCM treatments on fractures involve topical applications of herbal pastes. These treatment regimens have adopted by Chinese for thousands of years. The formulae of these herbal medicines are very often complicated and can include ten or more herbs. More importantly, these herbal formulations have not been well-accepted worldwide due to the serious lack of relevant evidence-based scientific supports and good systemic documentation of the clinical data. The aim of this study are to provide simplified herbal composition with evidence-based scientific data to verify the efficacy thereof on promotion of bone healing in vitro and in vivo.

Topical paste of TCM has been widely used in the therapy of bone and related injuries. In topical drug delivery, transdermal transport of herbal ingredients is the key factor for effective treatment. However, poor water solubility of active herbal ingredients creates a bottleneck in diffusion across the skin, and thus prominently limits the bioavailability and effectiveness of the topical medication. There is a need for a transdermal system for effective transport of active herbal ingredients in order to improve the bioavailability and effectiveness of topical herbal therapy.

### SUMMARY OF INVENTION

The present invention provides a herbal composition comprises hydrophilic extract and hydrophobic extract of at least one herb, a nano-emulsion that is capable of dispersing and dissolving said herbal composition, a matrix-type transdermal patch that is capable of transferring said herbal composition across the skin, method of preparing said herbal composition, nano-emulsion and matrix-type transdermal patch and the use thereof for treating traumatic injuries in tissues and bones.

In one aspect, the present invention is a herbal composition comprising extracts of Carthami Flos (CF), Dipsaci Radix (DR), Notoginseng Rhizoma (NR) and Rhei Rhizoma (RR) extracted by a hydrophilic and a hydrophobic solvent, and optionally a pharmaceutical acceptable carrier, wherein the herbal composition excludes extracts of Fructus Gardeniae (FG) and Sambucus Williamsii (SW).

In one embodiment, the present invention is a herbal composition consisting essentially of extracts of Carthami Flos (CF), Dipsaci Radix (DR), Notoginseng Rhizoma (NR) and Rhei Rhizoma (RR) extracted by a hydrophilic and a hydrophobic solvent, and optionally a pharmaceutical acceptable carrier.

In another embodiment, said hydrophilic solvent is distilled water and said hydrophobic solvent comprises alcohol. In another embodiment, the hydrophobic solvent comprises ethanol, for example, 95% aqueous ethanol.

In yet another embodiment, weight ratio of dry CF, DR, NR and RR is 0.8-1.2:1-1.5; 0.8-1.2:1-2. In another embodiment, the weight ratio of CF, DR, NR and RR is 1:1:1:1 to 1:1.5:1:2. The percentage by weight of said hydrophilic extract to the percentage by weight of said hydrophobic extract of the herbal composition is 10:1 to 7:1. In yet another embodiment, the percentage by weight of said hydrophilic extract to said hydrophobic extract is 30%:3% to 42%:6%, preferably 35%:4.3% to 39.8% to 5% of said herbal composition.

In another aspect of the present invention, the present herbal composition is prepared according to the following steps:

a) providing dry herbs Carthami Flos (CF), Dipsaci Radix (DR), Notoginseng Rhizoma (NR) and Rhei Rhizoma (RR), optionally cutting CF, DR, NR and RR into pieces;

b) soaking the CF, DR, NR and RR individually or jointly in a hydrophilic solvent and/or heating the CF, DR, NR and RR in the hydrophilic solvent under reflux to obtain a hydrophilic extract, optionally filtering, concentrating and/or freeze-drying the hydrophilic extract;

c) soaking the dry herbs of a) or residue from b) in a hydrophobic solvent and/or heating the dry herbs of a) or residue from b) in the hydrophobic solvent under reflux to obtain a hydrophobic extract, optionally filtering, concentrating and/or freeze-drying the hydrophobic extract; and

d) formulating the herbal composition from the hydrophilic extract and the hydrophobic extract, and at least one pharmaceutically acceptable carrier.

In another embodiment, the present herbal composition is prepared according to the following steps:

a) providing dry herbs of Carthami Flos (CF), Dipsaci Radix (DR), Notoginseng Rhizoma (NR) and Rhei Rhizoma (RR), optionally cutting CF, DR, NR and RR into pieces;

b) soaking the CF, DR, NR and RR individually or jointly in a hydrophobic solvent and/or heating the CF, DR, NR and RR in the hydrophobic solvent under reflux to obtain a hydrophobic extract, optionally filtering, concentrating and/or freeze-drying the hydrophobic extract;

c) soaking the dry herbs of a) or residue from b) in a hydrophilic solvent and/or heating the dry herbs of a) or residue from b) in the hydrophilic solvent under reflux to obtain a hydrophilic extract, optionally filtering, concentrating and/or freeze-drying the hydrophilic extract; and

d) formulating the herbal composition from the hydrophilic extract and the hydrophobic extract, and at least one pharmaceutically acceptable carrier.

In another embodiment, the present herbal composition can be, but not limited to, paste, cream, transdermal patches or nano-emulsion.

In another aspect, the present invention relates to an oil-in-water (o/w) or water-in-oil (w/o) type nano-emulsion. The nano-emulsion comprises an aqueous phase, an organic phase and a surfactant. Said aqueous phase comprises hydrophilic extract of one or more herbal materials. Said organic phase comprises hydrophobic extract of said one or more herbal materials.

In one embodiment, the nano-emulsion is an oil-in-water type nano-emulsion.

In another embodiment, said aqueous phase or organic phase is a droplet of 10-200 nm in diameter. In another embodiment, said aqueous phase or organic phase is a droplet of 10-50 nm in diameter; and/or said aqueous phase is 60-90% of said nano-emulsion by weight, preferably 75-85%; and/or said organic phase is 2-20% of said nano-emulsion by weight, or 2-8% of said nano-emulsion by weight; and/or said surfactant is 4-40% of said nano-emulsion by weight, or 10-22%.

Type of the nano-emulsion is determined by a dispersed phase being an aqueous or organic phase. Said dispersed phase may be water or oil droplet of 10-200 nm, or 10-50 nm in diameter.

In one embodiment, the aqueous phase of the present nano-emulsion is formed by an aqueous solvent, wherein said aqueous solvent is water; and/or the organic phase of the present-nano-emulsion is formed by an organic phase solvent, wherein said organic phase solvent is an oil that comprises Sefsol-218, Capryol-90, triglyceride, Myritol-318, limonene, liquid paraffin or ethanol, or a combination thereof; and/or wherein said surfactant has a hydrophilic-lipophilic balance (HLB) value in a range of 8-18. Examples of surfactant include, but are not limited to, Labrasol, Cremophor EL, Tween 20, Tween 60 and Tween 80. In one embodiment, the surfactant is Cremophor EL or Tween 20.

In one embodiment, the present nano-emulsion comprises the hydrophilic extract and hydrophobic extract of the herbal composition.

In one embodiment, the hydrophilic extract of the present nano-emulsion by weight (w/w) is 5-10%; and the hydrophobic extract of the present nano-emulsion by weight (w/w) is 22-30%.

In another aspect, the present invention relates to method of preparing the oil-in-water or water-in-oil nano-emulsion, said method comprises:

1) extracting hydrophilic extract from herbs using a hydrophilic solvent, wherein said hydrophilic solvent is water;

2) extracting hydrophobic extract from herbs using a hydrophobic solvent, wherein said hydrophobic solvent is alcohol, or 95% ethanol;

3) dissolving said hydrophilic extract obtained in 1) in an aqueous solvent to form an aqueous phase, and dissolving said hydrophobic extract obtained in 2) in an organic phase solvent to form an organic phase;

4) mixing said aqueous phase and said organic phase with a surfactant to form a pre-mixture;

5) homogenizing said pre-mixture under 12,000-30,000 rpm for 10-30 mins to form the nano-emulsion.

In another aspect, the present invention relates to a transdermal patch. The transdermal patch comprises a backing layer, a matrix layer and a liner layer. Said matrix layer comprises the oil-in-water or water-in-oil nano-emulsion of the present invention and an adhesive material. The nano-emulsion is 1-25% by weight of the matrix layer; wherein said adhesive material is selected from the group consisting of Carbomer 980, Carbopol U20, Carbopol HV-805EG, carboxymethylcellulose sodium, gelatin, poly(acrylic acid sodium salt), Poly(acrylic acid sodium salt) NP700, Polyvinylpyrrolidone 10, Polyvinvylpyrrolidone K90 and a combination thereof. In one embodiment, the adhesive material is selected from poly(acrylic acid sodium salt), Poly(acrylic acid sodium salt) NP700, Carbopol HV-805EG, Polyvinylpyrrolidone 10, Polyvinvylpyrrolidone K90 and a combination thereof, and wherein the adhesive material is 10-15% by weight of the matrix layer.

In one embodiment, said matrix layer further comprises a crosslinking agent, a humectant, fillers, scaffold, or a pH controlling agent.

In one embodiment, 1-25% of the total mass of the matrix layer is the nano-emulsion, 10-15% is the adhesive material, 0.01-0.8% is the crosslinking agent, 15-25% is the humectant, 1-10% is the scaffold or filler and a pH controlling agent that maintains the matrix layer at a pH 5-6.

In another aspect, the present application provides the use of a herbal composition, a nano-emulsion and a transdermal patch in treating traumatic injuries.

In one embodiment, said traumatic injuries include tissue and bone injuries. For example, traumatic musculoskeletal injuries, soft tissues injuries and fractures.

### BRIEF DESCRIPTION OF DRAWINGS

FIG. 1 shows effects of hydrophilic extract (FIG. 1A) and hydrophobic extract (FIG. 1B) of CR, DR, NR and RR (collectively referred to as CDNR) in accordance with the present invention on NO production in LPS-induced RAW264.7 cells

FIG. 2 shows effects of CDNR hydrophilic extract (FIG. 2A) and CDNR hydrophobic extract (FIG. 2B) in accordance with the present invention on viability and proliferation of UMR-106 osteoblast cells

FIG. 3 shows changes in biomechanical properties of damaged bone tissue after six weeks treatment of the herbal composition of the present invention: yield strength (FIG. 3A); work done at yield strength (FIG. 3B); ultimate strength (FIG. 3C); work done at ultimate strength (FIG. 3D); failure strength (FIG. 3E) and work done at failure strength (FIG. 3F)

FIG. 4 shows the elevated rate of transdermal diffusion of the tested transdermal patch relative to the herbal paste control; markers diffused in the receiving medium (FIG. 4A) and remained in pig skin (FIG. 4B)

FIG. 5 shows a diagram of a matrix-type transdermal patch of the present invention

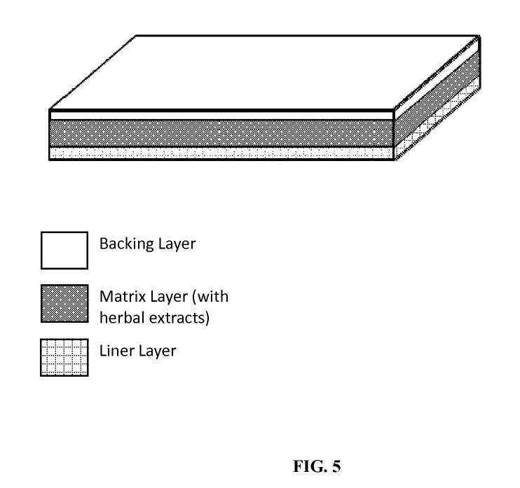

### DETAILED DESCRIPTION OF INVENTION

The following specific embodiments of the present invention is described more fully and its many additional advantages to the present invention and a variety of additional advantages of the invention would be clear to those skilled in the art.

One aspect of the present invention provides a herbal composition, said composition comprises hydrophilic extract and hydrophobic extract of Carthami Flos (CF), Dipsaci Radix (DR), Notoginseng Rhizoma (NR) and Rhei Rhizoma (RR), and optionally a pharmaceutically acceptable carrier, wherein said composition excludes extracts of Fructus Gardeniae (FG) and Sambucus Williamsii (SW). In one embodiment, the herbal composition of the present invention consisting essentially of hydrophilic extract and hydrophobic extract of Carthami Flos (CF), Dipsaci Radix (DR), Notoginseng Rhizoma (NR) and Rhei Rhizoma (RR), and optionally a pharmaceutically acceptable carrier.

Said hydrophilic and hydrophobic extracts can be any pharmaceutically acceptable hydrophilic solvent or any pharmaceutically acceptable hydrophobic solvent that are suitable for extracting hydrophilic and hydrophobic components of CF, DR, NR and RR, so as to fully obtain hydrophilic and hydrophobic components of the medicinal herbs having pharmaceutically active ingredients.

In one embodiment, said suitable hydrophilic solvent is water, preferably distilled water and said suitable hydrophobic solvent is 95% ethanol.

In another embodiment, weight ratio of dry CF, DR, NR and RR is 0.8-1.2:1-1.5; 0.8-1.2:1-2. In another embodiment, the weight ratio of CF, DR, NR and RR is 1:1:1:1 to 1:1.5:1:2. The percentage by weight of said hydrophilic extract to the percentage by weight of said hydrophobic extract of the herbal composition is 10:1 to 7:1. In yet another embodiment, the percentage by weight of said hydrophilic extracts to said hydrophobic extracts is 40%:4% to 35%:5%, preferably 35%:4.3% to 39.8% to 5% of said herbal composition.

The present herbal composition is topically administered to treat traumatic injuries. Said traumatic injuries comprises tissue damages (e.g. soft tissues) or bone damage. For example: traumatic musculoskeletal injuries, such as fractures.

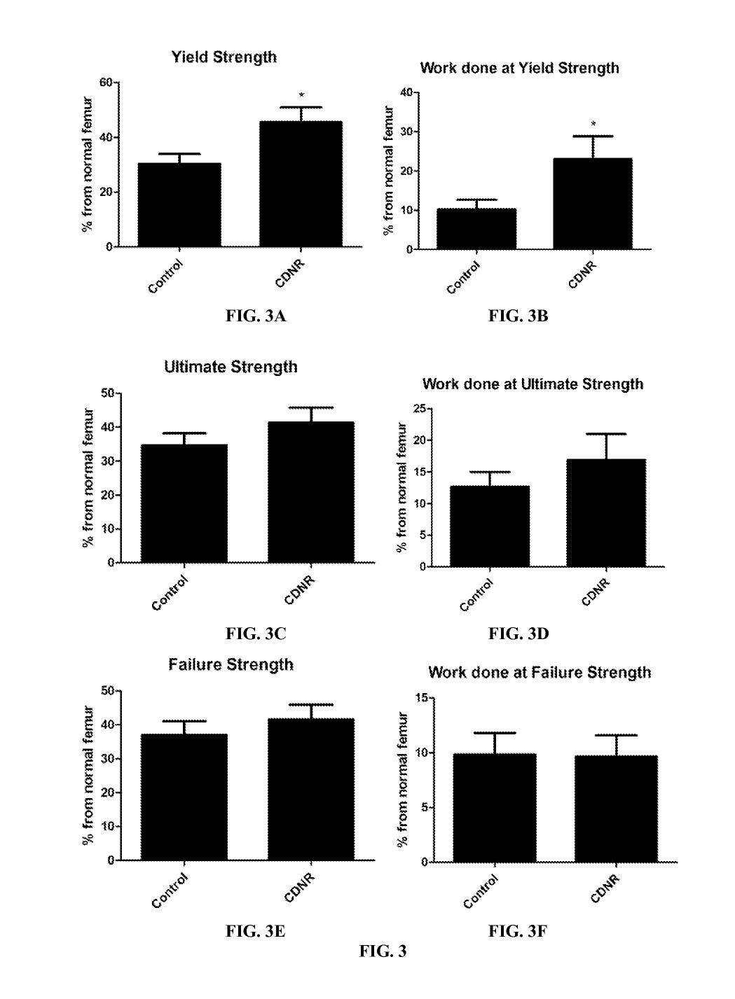

Said traumatic injuries treatment is based on the capability of the present herbal composition in promoting bone healing. As evidenced by the below working examples, the hydrophilic extract and hydrophobic extract of the present herbal composition synergistically control and coordinate inflammation and bone regeneration, promote cortical bone repair and healing of damaged bone tissues in bone fractures. The medicinal herb of the present invention undergo extraction as exemplified in Example 1, wherein the hydrophilic extract and the hydrophobic extract comprises the hydrophilic active ingredient and hydrophobic active ingredient, respectively. The two type active ingredients synergistically promote healing of traumatic bone tissue damage, as evidenced in Example 2. As seen in FIG. 3, the herbal composition of the present invention significantly increases biomechanical strength of bones, despite being a simplified composition. Additionally, CDNR extract of the present herbal composition associates with significantly low side-effects in organism, e.g. human, thereby reducing any adverse risks during treatment. The medicinal herbs of the present herbal composition have clear chemical indexes, enabling quality control of the herb and transdermal research.

Another aspect of the present invention provides method of preparing said herbal composition, wherein said method comprises:

a) providing dry herbs of Carthami Flos (CF), Dipsaci Radix (DR), Notoginseng Rhizoma (NR) and Rhei Rhizoma (RR), optionally cutting CF, DR, NR and RR into pieces;

b) soaking the CF, DR, NR and RR individually or jointly in a hydrophilic solvent and/or heating the CF, DR, NR and RR in the hydrophilic solvent under reflux to obtain a hydrophilic extract, optionally filtering, concentrating and/or freeze-drying the hydrophilic extract;

c) soaking the dry herbs of a) or residue from b) in a hydrophobic solvent and/or heating the raw material of a) or residue from b) in the hydrophobic solvent under reflux to obtain a hydrophobic extract, optionally filtering, concentrating and/or freeze-drying the hydrophobic extract; and

d) formulating the herbal composition from the hydrophilic extract and the hydrophobic extract, wherein optionally the herbal composition is formulated with at least one pharmaceutically acceptable excipient.

Steps b) and c) are sequentially exchangeable. This means that hydrophobic extract can be obtained before the hydrophilic extract. In a preferred embodiment, the hydrophilic extract is obtained first. Filtration step is used to remove solid residue.

The time for soaking the herbal material and/or heating the herbal material under reflux must be long enough to fully extract the medicinal herbal active ingredient. Said four raw herbal materials may be soaked under a hydrophilic solvent, such as distilled water, for at least 1 hour; may be heated under distilled water under reflux for at least 2 hours, preferably heated under reflux for at least 2 hours twice, in order to fully extract the hydrophilic active ingredient. Residues of the hydrophilic extract are heated under reflux with a hydrophobic solvent, such as 95% ethanol, for at least 2 hours in order to fully extract the hydrophobic ingredient. Extraction conditions of different herbal materials can be readily determined by one skilled in the art.

Said formulating step is determined by the mode of administration of the herbal composition. For example: said hydrophilic extract and said hydrophobic extract can be simply mixed together, or with any suitable pharmaceutically acceptable excipient in order to formulate a composition suitable for different modes of administration. For topical administration, the herbal composition is formulated as nano-emulsion as described herein below, in order to improve transdermal transport of active herbal ingredients, and thereby improving the bioavailability and effectiveness of the therapy.

In one embodiment, said hydrophilic and hydrophobic extracts are filtered, concentrated, freeze-dried, followed by mixing with 50% ethanol solution to obtain a paste for topical administration.

Another aspect of the present invention provides a nano-emulsion comprises an aqueous phase, an organic phase, and a surfactant, wherein said aqueous phase comprises hydrophilic extract of herbal materials and/or said organic phase comprises hydrophobic extract of the herbal materials. Depending on the type of pharmaceutical active ingredient and concentration thereof, said nano-emulsion can be oil-in-water (o/w) or water-in-oil (w/o) type nano-emulsion, thus a dispersed phase can be aqueous or organic. According to laser scattering measurement, Said dispersed phase may be water or oil droplet of 10-200 nm, or 10-50 nm in diameter. Type of the nano-emulsion is determined by a dispersed phase being an aqueous or organic phase.

One skilled in the art can readily determine content of the aqueous phase and organic phase according to the type of nano-emulsion. For example, said aqueous phase is 60-90% of said nano-emulsion by weight, preferably 75-85%; and/or said organic phase is 2-20% of said nano-emulsion by weight, or 2-8% of said nano-emulsion by weight; and/or said surfactant is 4-40% of said nano-emulsion by weight, or 10-22%. Any pharmaceutically acceptable aqueous solvent, such as water, readily know in the art may be suitable for use as the aqueous solvent of the aqueous phase.

Any pharmaceutically acceptable organic solvent, readily known in the art may be suitable for use as the organic phase solvent of the organic phase. For example, Sefsol-218, Capryol-90, triglyceride, Myritol-318, limonene, liquid paraffin or ethanol, or a combination thereof.

Surfactant may be added to reduce the surface tension between the continuous phase and dispersed phase and thereby forming an emulsion. An appropriate surfactant is selected according to the desired nano-emulsion type (o/w or w/o type). Surfactant that is favorable for the formation of o/w type emulsion is a nonionic surfactant having a hydrophilic-lipophilic balance (HLB) value in a range of 8-19. Examples of surfactant that is favorable for the formation of o/w type emulsion include, but are not limited to, Labrasol, Cremophor EL, Tween 20, Tween 60 and Tween 80. In one embodiment, the surfactant is Cremophor EL or Tween 20. The surfactant is 4-40% or 10-20% by weight of the nano-emulsion.

It is important that the herbal extract is completely dissolved in the aqueous solvent or the organic solvent to ensure sufficient homogenization. Hydrophilic extract and hydrophilic extract of the herbal extract may be obtained by the aforementioned methods and any suitable methods known to a skilled in the art. The herbal extract and volume of solvent thereof is determined by the use of the herbal composition, disease to which the herbal composition targets, type of the extract component, extraction rate, solubility of the herbal extract in hydrophilic and hydrophobic solvents and the like which can be readily determined by one skilled in the art.

In one embodiment of the present invention, wherein a hydrophobic solvent such as an alcohol, like ethanol is used for extraction, due to the nature of the hydrophobic solvent used, the hydrophobic extract obtained from such hydrophobic solvent comprises hydrophilic component and hydrophobic component of the herbs. In another embodiment, the herbal material is first extracted by hydrophilic solvent, followed by extraction by hydrophobic solvent. During extraction of the hydrophobic solvent, the remaining hydrophilic component in the herbal material is being extracted together with the hydrophobic component. Therefore, a portion of the hydrophobic extract is hydrophilic. The hydrophilic portion of the hydrophobic extract is found within the aqueous phase of the resultant nano-emulsion. In one embodiment, the hydrophilic extract of the present nano-emulsion is 5-10 wt %; and the hydrophobic extract of the present nano-emulsion is 22-30 wt %.

In another embodiment, the nano-emulsion is an o/w type nano-emulsion, wherein the aqueous phase of the nano-emulsion comprises hydrophilic extract of the herbal composition (CF, DR, NR and RR) and the organic phase of the nano-emulsion comprise hydrophobic extract of the herbal composition. In another embodiment, a first solvent for forming said aqueous phase is water; and/or a second solvent for forming said organic phase is Capryol-90. Said o/w type nano-emulsion may be formed, e.g. aqueous phase, organic phase, hydrophilic extract and hydrophobic extract, formulating said o/w type nano-emulsion, methods as described above.

In another embodiment, the present nano-emulsion comprises 74.4-84.8 wt % of aqueous phase, 3.8-3.9 wt % organic phase and 11.4-21.7 wt % surfactant.

In yet another embodiment, the present nano-emulsion comprises 74.4-84.8 wt % aqueous phase, wherein the aqueous solvent is water; 3.8-3.9 wt % organic phase, wherein the organic solvent is Capryol-90; and 11.4-21.7 wt % Tween 20 as the surfactant. In another embodiment, the hydrophilic extract in said aqueous phase is 5-10% of the nano-emulsion, the hydrophobic extract in said organic phase is 22-30% of the nano-emulsion. The present nano-emulsion provides an effective way in delivering the hydrophilic and hydrophobic extract of the herbal material by significantly increasing the transdermal diffusion rate of thereof.

Another aspect of the present invention provides a method of preparing said nano-emulsion, said method comprises:

a) extracting a hydrophilic extract from herbs using a hydrophilic solvent, wherein said hydrophilic solvent is water;

b) extracting a hydrophobic extract from herbs using a hydrophobic solvent, wherein said hydrophobic solvent is alcohol, or 95% ethanol;

c) dissolving said hydrophilic extract obtained in a) in an aqueous solvent to form an aqueous phase, and dissolving said hydrophobic extract obtained in b) in an organic solvent to form an organic phase;

d) mixing said aqueous phase and said organic phase with a surfactant to form a pre-mixture;

e) homogenizing said pre-mixture under 12,000-30,000 rpm for 10-30 mins to form the nano-emulsion.

In another embodiment, the herbal hydrophilic extract and the herbal hydrophobic extract are completely dissolved in the aqueous solvent and organic phase solvent used to form the aqueous phase and organic phase of the nano-emulsion, respectively. Thus, an aqueous phase comprises the hydrophilic herbal extract and an organic phase comprises the hydrophobic herbal extract are formed.

Optionally said herbal hydrophilic extract and herbal hydrophobic extract are filtered, concentrated and/or freeze-dried. Optionally, said herbal hydrophilic and hydrophobic extracts are freeze-dried powder.

In another embodiment, said herbal hydrophilic extract and said herbal hydrophobic extract are the hydrophilic extract and hydrophobic extract of the aforementioned present herbal composition, i.e. hydrophilic and hydrophobic extracts of CF, DR, NR and RR. Said extracts are optionally filtered, concentrated (e.g. by reduced pressure) and freeze-dried to become a powder form.

In one embodiment of the present method of preparing a nano-emulsion, a surfactant is first mixed with the aqueous phase, followed by mixing with the organic phase to form a pre-mixture. Homogenization of the pre-mixture can be done using mechanical homogenizer, e.g. 12,000-30,000 rpm for 10-30 mins, or 24,000-30,000 rpm for 20-30 mins, to obtain a nano-emulsion.

The nano-emulsion obtained can be further processed by high pressure homogenization in order to further refine the homogeneity. The pressure can be set at 200-800 bar, average cycle number can be 2-10 times.

The present nano-emulsion can significantly improve the transdermal diffusion of pharmaceutical active ingredient, thereby enhancing the bioavailability of the active ingredient and leading a traumatic injury treatment with high efficacy, as compared to existing nano-emulsion. Furthermore, the aqueous and organic phases of the present nano-emulsion can comprise multiple types of active ingredients.

The nano-emulsion of the present invention is applicable to any herbal formulation having active ingredient with a hydrophilic and/or hydrophobic component, in particular, the herbal composition of the present invention.

Another aspect of the present invention provides a transdermal patch. The present transdermal patch comprises a backing layer, a matrix layer and a liner layer. Said matrix layer comprises the oil-in-water or water-in-oil nano-emulsion of the present invention and an adhesive material. The nano-emulsion includes 1-25% by weight of the matrix layer; wherein said adhesive material is selected from the group consisting of Carbomer 980, Carbopol U20, Carbopol HV-805EG, carboxymethylcellulose sodium, gelatin, poly(acrylic acid sodium salt), Poly(acrylic acid sodium salt) NP700, Polyvinylpyrrolidone 10, Polyvinvylpyrrolidone K90 and a combination thereof. In one embodiment, the adhesive material comprises poly(acrylic acid sodium salt), Poly(acrylic acid sodium salt) NP700, Carbopol HV-805EG, Polyvinylpyrrolidone 10 and Polyvinvylpyrrolidone K90 of 10-15% by weight.

In one embodiment, said matrix layer further comprises at least one of a crosslinking agent, a humectant, fillers, scaffold, or a pH controlling agent.

A crosslinking agent is usually used for forming a matrix network of said matrix layer. Examples of suitable crosslinking agents include, but are not limited to, aluminum chloride, citric acid, dihydroxyaluminum aminoacetate and poly(vinyl alcohol). In one embodiment, where poly(acrylic acid sodium salt) is the adhesive material, dihydroxyaluminum aminoacetate is preferably used as the crosslinking agent. The crosslinking agent is 0.01-0.8 wt % of the matrix layer.

Fillers or scaffold is usually added to support the matrix network of the matrix layer. Examples of said fillers or scaffold include, but are not limited to, calcium carbonate, kaolin, silicon dioxide and zinc oxide. In one embodiment, kaolin is used as fillers or scaffold. The filler or scaffold is about 1-10 wt % of the matrix layer.

Said matrix layer further comprises one or more humectant to maintain the patch moist. Examples of humectant include, but are not limited to, glycerin, propylene glycol, PEG-400, D-sorbitol and Tween 80. In one embodiment, the matrix layer comprises glycerin and D-sorbitol. The humectant is 10-40% or 15-25% by weight of the matrix layer.

Said matrix layer further comprises a pH controlling agent, e.g. tartaric acid. The matrix layer is controlled at pH5-6.

In one embodiment, said matrix layer comprises 1-25 wt % of the nano-emulsion, 10-15% of the adhesive material, 0.01-0.8% of the crosslinking agent, 15-25% of humectant, 1-10% of filler or scaffold and a pH controlling agent that maintain the matrix layer at pH 5-6.

In another embodiment, the present matrix layer comprises 17.4%-18.4 wt % nano-emulsion, 12.4-12.7 wt % adhesive material, 0.4 wt % crosslinking agent, 19-19.7 wt % humectant, 4.6 wt % scaffold or filler, 0.2 wt % of a pH controlling agent that maintains pH 5-6 and remaining is water.

In yet another embodiment, the present matrix layer comprises 17.4-18.4 wt % nano-emulsion; 8.1 wt % poly(acrylic acid sodium salt), 2.9 wt % Polyvinylpyrrolidone 10 and 1.4 wt % Carbopol U20 as adhesive material or 8.1% poly(acrylic acid sodium salt), 0.3 wt % Poly(acrylic acid sodium salt) NP700, 2.9 wt % Polyvinylpyrrolidone 10 and 1.4 wt % Carbopol HV-805EG as the adhesive material; 0.4 wt % aluminum chloride or 0.4 wt % dihydroxyaluminum aminoacetate as the crosslinking agent; 5.8 wt % glycerin and 13.9 wt % D-sorbitol as humectant; 4.6 wt % kaolin as scaffold; 0.2 wt % tartaric acid as pH controlling agent to control pH of 5-6 and remaining is water.

In another aspect, the nano-emulsion of the matrix layer is as defined in the present application. The matrix layer of the present transdermal patch is particularly effective in transporting herbal hydrophilic and hydrophobic extract across skin, significantly increases medicinal herbs (such as CF, DR, NR, RR and combination thereof) bioavailability and transdermal diffusion rate. The backing layer of the present patch functions as an inert layer. The liner layer of the present patch covers and protects said matrix layer.

The backing layer can be a polyethylene-based film or multiple polyester-based laminates combined to form a single web. Examples of backing layer include, but are not limited to, CoTran 9720, Co Tran 9722, Scotchpak 1109, Scotch 9723, Scotchpak 9730 and Scotchpak 9735. The polyester-based film useful for the present invention does not react with the matrix layer.

Said liner layer is occlusive that is low moisture vapor transmission and compatible with said matrix layer to protect said matrix layer.

The transdermal patch of the present invention is prepared as follows: uniformly coating a matrix or matrix web that forms a matrix layer onto a backing layer; drying at 65-100° C. for 6-24 hours, or 70-80° C. for 10-16 hours; placing a liner layer on said matrix layer for protection after cooling to form said transdermal patch.

The present invention relates to the above mentioned herbal composition, a nano-emulsion comprises said herbal composition and a transdermal patch comprises said herbal composition for use in the manufacture of a medicament for the treatment of traumatic injuries. The herbal composition, the nano-emulsion and the transdermal patch of the present invention each comprise hydrophilic and hydrophobic herbal extracts.

In one embodiment, said traumatic injuries include tissues (e.g. soft tissues) damage or bone damage; e.g. traumatic musculoskeletal injuries, such as bone fractures.

The herbal composition and nano-emulsion comprises the herbal composition are administered topically.

Due to the capability of the nano-emulsion of the present invention to promote transdermal delivery of pharmaceutically active ingredient by diffusion, when the hydrophilic and hydrophobic herbal extract of the present o/w or w/o type nano-emulsion and the transdermal patch of the present invention is administered topically, transdermal diffusion of the active ingredients in the herbal extracts is promoted. Thus, bioavailability of the active ingredients increases and thereby improving the efficacy in traumatic injuries treatment.

Hereinafter the present invention will be described in detail with reference to specific embodiments. However, one skilled in the art will appreciate, the particular embodiments only serve to explain the present invention more clearly, in any case should not be construed as a limitation of the present invention. Unless otherwise specified, all reagents and equipment are commercially available and all contents are by weight.

### EXAMPLES

The herbs CF, DR, NR and RR used in the examples below have been authenticated using thin-layer chromatography as described in Chinese Pharmacopoeia 2010. The herbs are purchased from a local herbal supplier in Hong Kong, and identities thereof are authenticated using the aforesaid methods.

### Example 1

### Preparation of Hydrophilic Extract and Hydrophobic Extract of CF, DR, NR and RR of the Present Herbal Composition

250 g raw materials of the four herbs are cut into small pieces (C:D:N:R in 1:1:1:1 w/w) and are soaked in 1.0 L distilled water for 1 hour. They are boiled twice for 2 hours under reflux and then the aqueous extract is collected and filtered through a piece of absorbent gauze. The residue is further boiled with 95% ethanol for 2 hours under reflux. The ethanol extract is collected and filtered again. Both of the aqueous and ethanol filtrate are then concentrated at 50° C. under reduced pressure separately, followed by lyophilizing into powder form in a freeze dry system (Freezone 12, Labconco, Mo., USA).

The extraction yields of aqueous [CDNR(aq)] and ethanolic [CDNR(e)] extracts are 39.8% w/w and 5.0% w/w, respectively.

A herbal paste of CDNR is prepared by mixing 19.5 g CDNR(aq) and 3.0 g CDNR(e) with 17 ml 50% ethanol for topical administration.

### Example 2

### Effects of Herbal Composition Comprises Hydrophilic Extract and Hydrophobic Extract of CDNR on Osteogenic Enhancement

Murine monocyte/macrophage RAW264.7, and rat osteoblast UMR-106 cells are purchased from American Type Culture Collection (ATCC; USA). CDNR (aq) and CDNR(e) from Example 1 are tested.

CDNR (e) inhibits NO production of LPS induced RAW264.7 cells

In order to determine the anti-inflammatory effect (i.e. inhibition of Lipposaccharide (LPS)-induced Nitric oxide (NO) production) of the present herbal composition, Murine macrophage Raw264.7 are grown at 37° C., 5% CO₂ in high-glucose DMEM medium (3500 mg/L; Life Technologies, USA) that comprises 10% v/v fetal bovine serum (Life Technologies, USA), 100 U/ml penicillin and 100 mg/L streptomycin (Life Technologies, USA).

Depending on the solubility, the DMEM medium or DMSO (1%, v/v) are used to dissolve CDNR (aq) to 0, 50, 100, 200 and 400 μl/mL, dissolve CDNR (e) to 0, 25, 50, 100 and 200 μl/mL. 1% DMSO has no effect on cell growth and NO production.

4×10⁶ cells/well of RAW264.7 are seeded onto 24 well plates, and cultured with various concentrations of CDNR (aq) or CDNR(e) for 24 hours. Each concentration is tested in triplicates. 1 μg/mL LPS are then added to each test and cultured for 24 hours. Supernatant are collected and Griess reagent are added to the test cells for 15 min. The absorbance of each well is measured under 540 nm in order to calculate the NO generated.

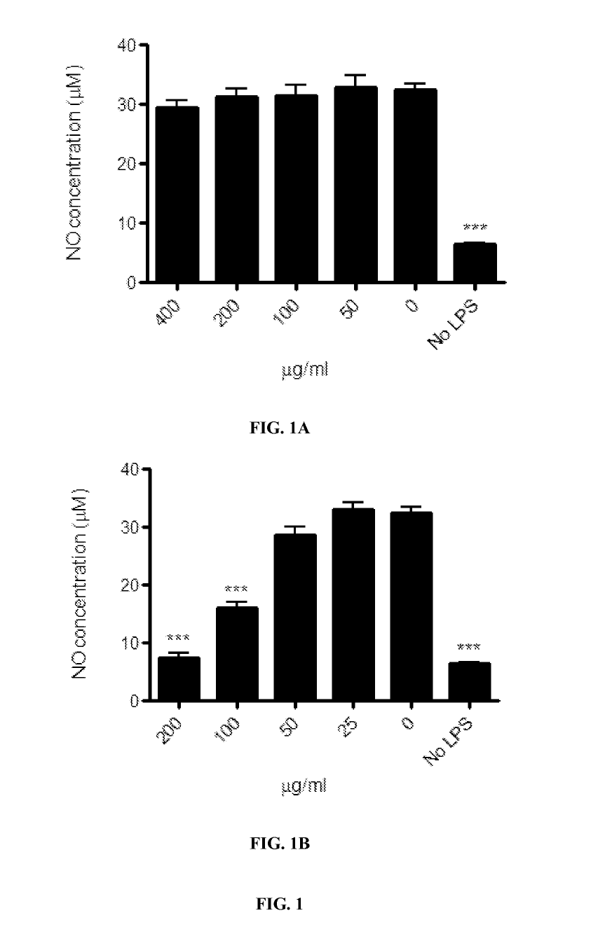

As seen in FIG. 1A and FIG. 1B, CDNR (aq) has no effect on NO production in LPS-induced RAW264.7. In contrast, CDNR (e), at 100 μl/mL and 200 μl/mL significantly suppress NO production in RAW264.7 by 51% and 77%, respectively (p<0.05) when compare to the baseline control group (i.e. no CDNR (e) or CDNR (aq) present). Therefore, the hydrophobic extract of CDNR has potent anti-inflammatory effect.

The herbal paste of Example 1 is also tested to determine the effect of hydrophilic and hydrophobic mixture on NO production. Similar inhibition results of NO production as CDNR(e) alone are observed. This indicates that hydrophilic herbal extract does not affect hydrophobic extract's anti-inflammatory action.

Hydrophilic CDNR Extract Promotes UMR-106 Cell Viability and Proliferation

Rat osteoblast UMR-106 are grown at 37° C., 5% CO₂ in high-glucose DMEM medium (3500 mg/L; Life Technologies, USA) that comprises 10% v/v fetal bovine serum (Life Technologies, USA), 100 U/ml penicillin and 100 mg/L streptomycin (Life Technologies, USA).

Depending on the solubility, the DMEM medium or DMSO (1%, v/v) are used to dissolve CDNR (aq) to 0, 6.25, 12.5, 25, 50 and 100 μl/mL, dissolve CDNR (e) to 0, 6.25, 12.5, 25, 50 and 100 μl/mL.

Effects of the herbal extracts on cell proliferation are measured using BrdU-ELISA kit (Roches, USA). 1000 cells/well of UMR-106 are seeded on to 96 well plate and grown until adhere to plate. Hydrophilic or hydrophobic herbal extracts of various concentrations are added and incubated for 24 hours. Each test is done in triplicates. 10 μl of BrdU working solution is added to each well (5 mg/ml stock solution are diluted to 6 μl/mL) and further incubated for 2 hours. Supernatant is then removed and cells are washed with wash buffer, anti-BrdU antibody is added and incubated for 120 min. Cell proliferation is measured according to the instructions appended to the BrdU-ELISA kit.

The effect of different concentrations of herbal extracts on cell proliferation is indicated as a ratio of cell proliferation having administered with the herbal extract to blank control group (with no herbal extract). Cell proliferation of the blank group is taken as 100%.

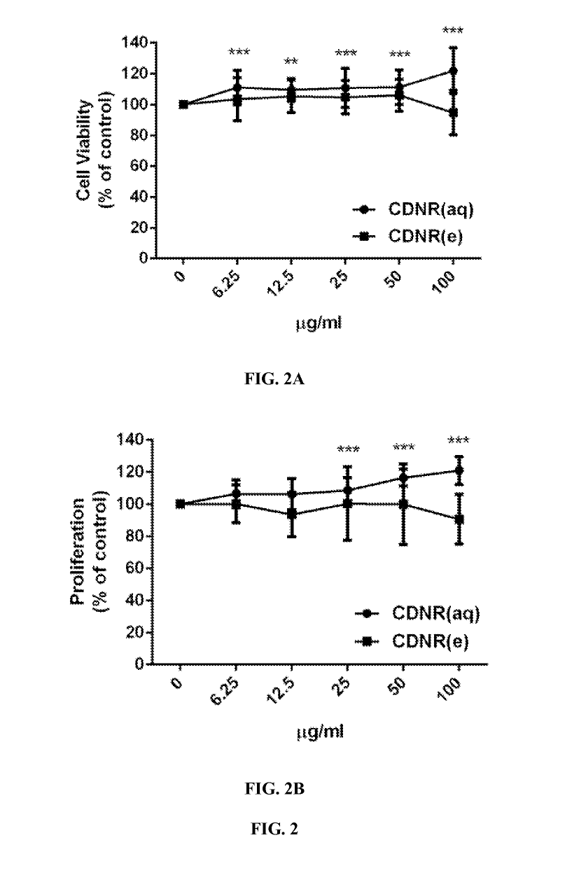

As seen in FIG. 2A and FIG. 2B, after treatment of 6.25-100 μl/mL CDNR (aq) for 24 hours, cell viability and proliferation of UMR-106 osteoblast cells has increased from 11% to 20% and 6% to 22%, respectively. Significant viability-enhancement and proliferative effect of CDNR (aq) are observed. On the other hand, 24 hours treatment of CDNR (e) has not imposed any cell viability and proliferation enhancement. CDNR (e) is shown to have no effect on cell viability and proliferation. It is shown that CDNR (aq) has potent effect in osteogenesis.

The herbal paste of Example 1 are also tested to determine the effect of hydrophilic and hydrophophobic mixture on cell viability and proliferation. Similar results as CDNR(aq) alone are observed. This indicates that hydrophobic herbal extract does not affect hydrophilic extract's osteogenesis action.

Evaluation of the Present Herbal Paste in Enhancing Bone Healing In Vivo

20 female Sprague-Dawley rats with age 15.2±1.41 months (mean±standard deviation) are obtained from the Laboratory Animal Service Centre of the Chinese University of Hong Kong (CUHK). All of them are housed in a temperature-controlled (25° C.) and light-controlled (12-h light/dark cycle) environment.

The rats are first anesthetized using a cocktail of 80 mg/kg ketamine and 8 mg/kg xylazine intramuscularly. On the left femur, two adjacent bilateral drill holes (2 mm in diameter each) are created using an electric drill on the mid-shaft of the femur through anterior-posterior approach. The two holes are bridged to form a 2 mm×4 mm defect using a dental milling bur. On the right tibia, a bilateral bone defect with 2.4 mm in diameter is made using the electric drill at the proximal metaphysis via medial-lateral approach. All the drilling processes are irrigated by 0.9% sterile saline and finally the drill-holes are flushed with plenty amount of the saline to discard bone fragments remained before the incisions are closed.

The rats are divided into two groups of ten. In the control group (Control), the left femur and the right tibia are covered with thin self-adhesive films without any treatment. In the herbal paste treatment group (CDNR), 0.5 ml CDNR paste from Example 1 is applied topically on the left femur and right tibia. The paste is protected from falling off and drying off by covering with a thin self-adhesive film. The whole treatment period is 6 weeks and all the films and paste are renewed at 2-day interval. After the rats had been euthanized at Day 42 and then both left and right femora of the rats are harvested. Excessive soft tissue is removed but the periosteum is preserved. Four-point bending test is performed using Hounsfield material testing machine (KM25, Redhill, United Kingdom). A load-cell with maximum 2500 N is mounted. The upper and lower supports span 8.0 and 20 mm, respectively. The drill-hole bone defect at the mid-shaft of the femur is located in the middle of the two upper supports and then the specimen is loaded at a constant speed of 5 mm/min in posterior-anterior approach until breakage. Load at yield is recorded for analysis. All the data of the drilled femur (left) are normalized with the normal femur (right). The results are expressed as the normalized percentage based on the normal femur.

As seen in FIG. 3A-FIG. 3F, prominent effect of the present CNDR composition on biomechanical properties of bone during healing is observed. In the cortical femur with drill-hole, 15% higher normalized yield strength and 13% of yield work done are found in CDNR group when compared with Control (p<0.05) after 42 days of treatment (FIG. 3A and FIG. 3B). CDNR treatment also demonstrates some beneficial effects in ultimate strength (FIG. 3C and FIG. 3D) and failure strength (FIG. 3E and FIG. 3F). The 4-point bending test illustrates that the present herbal paste significantly improves the biomechanical properties (bone strengths) of bone with drill-hole defect.

Based on the above results, it is shown that ethanolic extract of CDNR of the present herbal composition can effectively inhibit LPS induced NO production, thereby inhibit inflammation. “Anti-inflammation” is one of the key treatment principles to treat bone fracture in TCM theory. It aims to control the swelling and relief the pain and soreness from the fracture site and the surrounding soft tissue. In addition, water extract of CDNR of the present herbal composition can significantly enhance cell viability and proliferation of UMR-106, thereby enhance osteogenesis. Osteogenesis is an important factor for bone repair. It is especially effective during the reparative phase of fracture healing when endochondral ossification takes places and osteoblasts start to form new lamellar bone on the cartilaginous callus.

The result of the above biomechanical test is a strong evidence showing the in vivo promoting effect of the topical-use the present herbal paste on bone healing. The higher yield bending strength of femur in CDNR than in Control reveals that the treatment elevated the strength of the bone that can tolerate much more stress before permanent deformation (might be caused by micro-fracture) occurs. The above examples demonstrate that the present herbal composition comprising CDNR extracts are effective in regulating inflammation and bone regeneration in vitro. Its efficacy on promotion of cortical bone repair is also demonstrated in in vivo experiment. The present herbal composition also has similar effect when applied onto skin of the fracture.

Other ratios of CDNR paste (i.e. 0.8:1:0.8:1, 1.2:1.5:1.2:2 and 1:1.5:1:2), wherein weight ratio of hydrophilic extract to hydrophobic extract in the herbal paste is 30%:3% to 42%: 6%, including 35%:4.3% to 39.8%:5%, have also been tested. Similar anti-inflammatory and osteogenesis, cortical bone repair results that enhances traumatic bone repair and tissue repair effects have been obtained (data not shown).

Toxicity tests show that the present herbal composition is associated with very low toxicity to living organism (data not shown).

### Example 3.1

### Preparation of o/w Nano-Emulsion Comprising Hydrophilic and Hydrophobic Herbal Extracts

Hydrophilic CDNR extract and hydrophobic CDNR extract in powder form are obtained according to Example 1.

About 1 g of hydrophilic CDNR powder, about 1 g of hydrophobic CDNR powder, about 1 g of Capryol-90 and 7 g water are mixed thoroughly. Sonication is applied to order to dissolve the CDNR powder completely. The mixture is allowed to phase separate under room temperature. The top layer is collected as the organic phase while the bottom layer is the aqueous phase.

1.71 g of surfactant, Tween 20 is first mixed with 12.73 g of aqueous phase under gentle stirring by a magnetic stirrer. It is then combined with 0.56 g of organic phase to form a prehomogenized mixture with 5 minutes of sonication.

The prehomogenized mixture is then homogenized by the mechanical homogenizer at a rotational speed of 24000 rpm for 20 minutes to form a nano-emulsion of the present invention.

The nano-emulsion is characterized by laser light scattering (90 Plus/BI-MAS, Brookhaven Instruments Corporation) that the average particle size of emulsion oil droplets is 200 nm. The nano-emulsion consists of 3.8 wt % of organic phase, 11.4 wt % of surfactant and 84.8 wt % aqueous phase.

### Example 3.2

### Preparation of o/w Nano-Emulsion Comprising Hydrophilic and Hydrophobic Herbal Extracts

Aqueous phase and organic phase of CDNR herbal extract are obtained according to Example 3.1.

3.48 g of Tween 20 is first mixed with 11.9 g of aqueous phase under gentle stirring by a magnetic stirrer. The mixture is then combined with 0.62 g of organic phase to form a prehomogenized mixture with 5 minutes of sonication.

The prehomogenized mixture is homogenized by the mechanical homogenizer at a rotational speed of 30000 rpm for 30 minutes to form a nano-emulsion of the present invention.

The nano-emulsion was characterized by laser light scattering and the average particle size of emulsion oil droplets is 40 nm. The nano-emulsion consists of 3.9 wt % of organic phase, 21.7 wt % of surfactant and 74.4 wt % aqueous phase.

### Example 4.1

### Preparation of Transdermal Patch Comprising the Present Nano-Emulsion

7 g of poly(acrylic acid sodium salt), 4 g of kaolin, 0.35 g of dihydroxyaluminium aminoacetate, 5 g glycerin and 12 g of D-Sorbitol are mixed together to form phase A. 0.15 g of tartaric acid is added to 15 g of water to obtain 0.1 wt % tartaric acid solution; 2.5 g of Polyvinylpyrrolidone 10 is then dissolved in the 0.1 wt % tartaric acid solution to give phase B. 1.2 g of Carbopol U20 is swell in 24 g of water with high-speed stirring by a magnetic stirrer to give a gel-form liquid of phase C.

Phases B and C are first mixed together and stirred at a speed of 1400 rpm for 3 minutes, and further mixed with phase A. The mixed solution (Phase A, B and C) is filtered through a 0.5 mm sieve and then combined with 15 g of nano-emulsion prepared according to Example 3.1 under stirring at 800 rpm for 10 min so as to form matrix mixture of the matrix layer.

The matrix obtained composes of 8.1 wt % of poly(acrylic acid sodium salt), 4.6 wt % of kaolin, 0.4 wt % dihydroxyaluminium aminoacetate, 5.8 wt % glycerin, 13.9 wt % of D-Sorbitol, 0.2 wt % tartaric acid, 2.9 wt % of Polyvinylpyrrolidone 10, 1.4 wt % Carbopol U20, 17.4 wt % of nano-emulsion, and the rest is water.

The obtained matrix mixture is evenly spread onto a backing layer (Scotchpak 9735) and baked at 75° C. for 12 hours. A liner layer (Scotchpak 1022) is placed onto the matrix to protect the drug-in-adhesive matrix after cooling to obtain the transdermal patch. The thickness of the patch is 1.1 mm and the patch adhesive force is 0.33 N.

### Example 4.2

### Preparation of Transdermal Patch Comprising the Present Nano-Emulsion

7 g of poly(acrylic acid sodium salt), 0.25 g of Poly(acrylic acid sodium salt) NP-700, 4 g of kaolin, 0.35 g of dihydroxyaluminium aminoacetate and 12 g of D-Sorbitol are mixed together to form phase A. 0.15 g of tartaric acid is added to 15 g of water to obtain 0.1 wt % tartaric acid solution; 2.5 g of Polyvinylpyrrolidone 10 is then dissolved in the 0.1 wt % tartaric acid solution to give phase B. 1.2 g of Carbopol HV-805EG is swell in 24 g of water with high-speed stirring by a magnetic stirrer to give a gel-form liquid of phase C.

Phases B and C are first mixed together and stirred at a speed of 1400 rpm for 3 minutes, and further mixed with phase A. The mixed solution (Phase A, B and C) is filtered through a 0.5 mm sieve and then combined with 4.5 g glycerin and 16 g of nano-emulsion prepared according to Example 3.2 under stirring at 800 rpm for 10 min so as to form matrix mixture of the matrix layer.

The matrix obtained composes of 8.1 wt % of poly(acrylic acid sodium salt), 0.3 wt % of Poly(acrylic acid sodium salt) NP-700, 4.6 wt % of kaolin, 0.4 wt % dihydroxyaluminium aminoacetate, 13.8 wt % of D-Sorbitol, 0.2 wt % tartaric acid, 2.9 wt % of Polyvinylpyrrolidone 10, 1.4 wt % Carbopol HV-805EG, 5.2 wt % of glycerin, 18.4 wt % of nano-emulsion, and the rest is water.

The obtained matrix mixture is evenly spread onto a backing layer (Scotchpak 9735) and baked at 75° C. for 12 hours. A liner layer (Scotchpak 1022) is placed onto the matrix to protect the drug-in-adhesive matrix after cooling to obtain the transdermal patch. The thickness of the patch is 1.2 mm and the patch adhesive force is 0.62 N.

### Example 5

### The Present Nano-Emulsion Enhances Bioavailability in Transdermal Absorption

A vertical Franz diffusion cell (Hanson Research, USA) is used to carry out in vitro skin diffusion study. The diffusion cell has a 7 ml receiving cell with a diffusion area of 3.8 cm². 1.1 mm thick of pig skin sample is cut into 4.5-5 cm² sheet, and is sandwiched between two compartments of the diffusion cell, wherein the stratum corneum faces the donor compartment and the dermis layer faces the receptor compartment. The receiving medium is formed by physiological saline solution of (0.9% NaCl). The donor compartment is filled with the test sample or control sample. The test sample is the transdermal patch prepared according to Example 4.2 with a surface area of 4 cm²; the control sample is the herbal paste according to Example 1. Using external water circulation device, the diffusion cell is maintained at 37° C. to simulate temperature inside living organism, and maintain stirring at 600 rpm. After 24 hours, contents of the receiving cell and pig skin sample are collected, the solution from the receiving cell (i.e. receiving medium) and pig skin are detected for chemical markers found therein in order to analyze the transdermal diffusion rate of the test sample and control sample. The test is performed in triplicate.

Active ingredients of CF, DR, NR and RR (i.e. chemical markers thereof) are shown in the below Table 1:

| Herb | Chemical Marker |
|---|---|
| Notoginseng Rhizoma (NR) | Ginsenoside Rg1 (Rg1) |
| | Ginsenoside Rb1 (Rb1) |
| Dipsaci Radix (DR) | Asperosaponin VI (ASP6) |
| | Oleanolic Acid (OA) |
| Rhei Rhizoma (RR) | Emodin (Emo) |
| | Rhein (Rhe) |
| Carthami Flos (CF) | Hydrosafflower yellow A (HYA) |
| | Kaempferol (Kae) |

HPLC-ESI-MS is used to detect diffused markers and control sample. Agilent 1290 Infinity LC system (purchased from Agilent Technologies) having a vacuum device, a binary liquid pump, an autosampler, Agilent 6410 triple quadrupole (Triple Quad) LC/MS connected with a Agilent MassHunter computer software (Workstation) is used. Acquity UPLC HSS T3 1.8 μm (2.1 mm×150 mm) column is used and the Acquity UPLC is maintained a temperature of 40° C.

A gradient flow phase consisting of water (A) and acetonitrile (B) is used to separate 9 chemical markers of the test sample. The gradient condition is 0-3 min, 10-27% B; 3-5 min, 27-33% B; 5-12 min, 33-33% B; 12-13 min, 33-80% B; 13-16 min, 80-90% B; 16-20 min, 90-90% B. The flow rate is controlled at 0.5 mL/min, and injection volume of 204.

MS analysis using Agilent 6410 triple quadrupole LC/MS configured with ESI MS is carried out under negative ion mode and multiple reaction monitoring modes. Target ions of HYA is m/z 611.2->325.0; Rg1 is m/z 799.5->637.4; ASP 6 is m/z 927.5->603.3; Rb1 is m/z 1107.6->119.0; Kae is m/z 285.0->117.0; Emo is m/z 269.0->241.0; Rhe is m/z 283.0->239.0 and OA is m/z 455.3->407.4.

### Results

Table 2 below indicates percentage by weight of markers of test sample (nano-emulsion of transdermal patch) and control (herbal patch) in the receiving cell.

| | Herbal Paste | Transdermal Patch |
|---|---|---|
| Marker | (Control), wt % | (Test Sample), wt % |
| Rg1 | 1.1884 | 16.3635 |
| Rb1 | 0.0167 | 7.8820 |
| ASP6 | 0.1375 | 4.4904 |
| OA | 0.0000 | 0.0000 |
| Emo | 0.0091 | 1.8494 |
| Rhein | 0.2299 | 4.8924 |
| HYA | 2.0879 | 16.5104 |
| Kae | 6.5294 | 72.3590 |

Table 3 below indicates below indicates percentage by weight of markers of test sample (nano-emulsion of transdermal patch) and control (herbal patch) that have diffused to the dermis.

| | Herbal Paste | Transdermal Patch |
|---|---|---|
| Marker | (Control), wt % | (Test Sample), wt % |
| Rb1 | 0.1256 | 2.6792 |
| Rb1 | 0.1245 | 2.1507 |
| ASP6 | 0.0859 | 2.8010 |
| OA | 0.1253 | 5.8588 |
| Emo | 0.2773 | 1.3656 |
| Rhein | 0.1371 | 1.4277 |
| HYA | 0.0117 | 0.4772 |
| Kae | 0.7532 | 9.0852 |

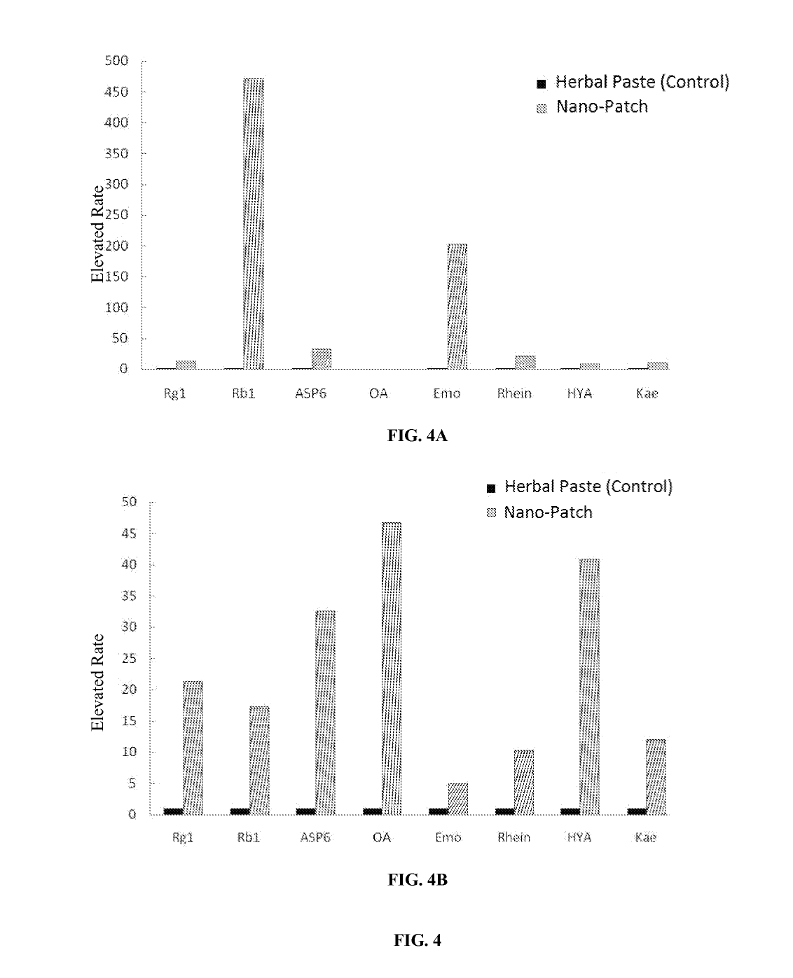

Results of Table 2 and Table 3 are graphically shown in FIG. 4A and FIG. 4B. The x-axis is the markers, y-axis is the increased rate of transdermal diffusion of the tested transdermal patch relative to the herbal paste control, in order to compare the diffusion properties of the test sample and control.

As seen FIG. 4A, OA cannot be detected in the receiving medium due to its low water solubility. For the other 7 markers, diffusion properties of HYA, Rg1, Rb1, ASP6, Emo, Rhe and Kae are elevated by 8 times, 14 times, 472 times, 32 times, 203 times, 21 times and 11 times, respectively. Similar results have been obtained where the four herbs are tested individually (Data not shown).

FIG. 4B shows the diffusion properties of the markers remained in the pig skin. The transdermal patch increases the diffusion properties of the markers. Diffusion properties of HYA, Rg1, Rb1, ASP6, Emo, Rhe, OA and Kae are elevated by 41 times, 21 times, 17 times, 33 times, 5 times, 11 times, 47 times and 12 times, respectively. Similar results have been obtained where the four herbs are tested individually (Data not shown). Evidently, the nano-emulsion and the transdermal patch comprising the nano-emulsion of the present invention are capable to increase the diffusion and absorption of pharmaceutical active ingredient. This elevated effect is particularly significant in certain active ingredients.

Further experiments show that the patch prepared as described in Example 4.2 promotes transdermal absorption when the patch is applied topically and increases therapeutic effect in bone fracture (data not shown).

As described above, although the invention is described herein with one or more embodiments, one of ordinary skill in the art will appreciate that the present invention can be modified without departing from the spirit and essence of the invention.

1. A herbal composition comprising hydrophilic extracts of Carthami Flos (CF), Dipsaci Radix (DR), Notoginseng Rhizoma (NR) and Rhei Rhizoma (RR) extracted by a hydrophilic solvent, hydrophobic extracts of Carthami Flos (CF), Dipsaci Radix (DR), Notoginseng Rhizoma (NR) and Rhei Rhizoma (RR) extracted by a hydrophobic solvent, and optionally a pharmaceutical acceptable carrier, wherein the herbal composition excludes extracts of Fructus Gardeniae (FG) and Sambucus Williamsii (SW).

2. The composition of claim 1, wherein the composition consists essentially of hydrophilic and hydrophobic extracts of Carthami Flos (CF), Dipsaci Radix (DR), Notoginseng Rhizoma (NR) and Rhei Rhizoma (RR), and a pharmaceutical acceptable carrier.

3. The composition of claim 1, wherein said hydrophilic solvent is distilled water and said hydrophobic solvent is 95% aqueous ethanol.

4. The composition of claim 1, wherein weight ratio of dry CF, DR, NR and RR is 0.8-1.2:1-1.5; 0.8-1.2:1-2 and percentage by weight of said hydrophilic extract to the percentage by weight hydrophobic extract of the herbal composition is 10:1 to 7:1.

5. The composition of claim 4, wherein the weight ratio of dry CF, DR, NR and RR is 1:1:1:1 to 1:1.5:1:2 and the percentage by weight of said hydrophilic extract to said hydrophobic extract is 30%:3% to 42%:6%.

6. The composition of claim 5, where percentage by weight of said hydrophilic extract to said hydrophobic extract is 35%:4.3% to 39.8% to 5% of said herbal composition.

7. A method of preparing the composition of claim 1, said method comprises:
a) providing dry herbs Carthami Flos (CF), Dipsaci Radix (DR), Notoginseng Rhizoma (NR) and Rhei Rhizoma (RR);
b) soaking the CF, DR, NR and RR individually or jointly in a hydrophilic solvent and/or heating the CF, DR, NR and RR in the hydrophilic solvent under reflux to obtain a hydrophilic extract, optionally filtering, concentrating and/or freeze-drying the hydrophilic extract;
c) soaking the dry herbs of a) or residue from b) in a hydrophobic solvent and/or heating the dry herbs of a) or residue from b) in the hydrophobic solvent under reflux to obtain a hydrophobic extract, optionally filtering, concentrating and/or freeze-drying the hydrophobic extract; and
d) formulating the herbal composition from the hydrophilic extract and the hydrophobic extract, with at least one pharmaceutically acceptable carrier.

8. A method of preparing the composition of claim 1, said method comprises:
a) providing dry herbs of Carthami Flos (CF), Dipsaci Radix (DR), Notoginseng Rhizoma (NR) and Rhei Rhizoma (RR), optionally cutting CF, DR, NR and RR into pieces;
b) soaking the CF, DR, NR and RR individually or jointly in a hydrophobic solvent and/or heating the CF, DR, NR and RR in the hydrophobic solvent under reflux to obtain a hydrophobic extract, optionally filtering, concentrating and/or freeze-drying the hydrophobic extract;
c) soaking the dry herbs of a) or residue from b) in a hydrophilic solvent and/or heating the dry herbs of a) or residue from b) in the hydrophilic solvent under reflux to obtain a hydrophilic extract, optionally filtering, concentrating and/or freeze-drying the hydrophilic extract; and
d) formulating the herbal composition from the hydrophilic extract and the hydrophobic extract, with at least one pharmaceutically acceptable carrier.

9. The composition of claim 1, wherein said composition is in a form selected from a paste, a cream, a transdermal patch and a nano-emulsion.

10. A nano-emulsion comprises an aqueous phase, an organic phase and a surfactant, wherein said aqueous phase comprises hydrophilic extract of one or more herbal materials and said organic phase comprises hydrophobic extract of said one or more herbal materials, and wherein the nano-emulsion is an oil-in-water (o/w) or water-in-oil (w/o) type nano-emulsion.

11. The nano-emulsion of claim 10, wherein said aqueous phase and organic phase is a droplet of 10-200 nm in diameter and said aqueous phase is 60-90% of said nano-emulsion by weight, said organic phase is 2-20% of said nano-emulsion by weight and said surfactant is 4-40% of said nano-emulsion by weight.

12. The nano-emulsion of claim 11, wherein said aqueous phase and organic phase is a droplet of 10-50 nm in diameter and said aqueous phase is 75-85% of said nano-emulsion by weight, said organic phase is 2-8% of said nano-emulsion by weight and said surfactant is 10-22% of said nano-emulsion by weight.

13. The nano-emulsion of claim 10, wherein the aqueous phase is formed by an aqueous solvent, wherein said aqueous solvent is water, said organic phase is formed by an organic phase solvent, wherein said organic phase solvent is an oil comprises Sefsol-218, Capryol-90, triglyceride, Myritol-318, limonene, liquid paraffin or ethanol, or a combination thereof; and said surfactant is a surfactant that has a hydrophilic-lipophilic balance (HLB) value in a range of 8-18.

14. The nano-emulsion of claim 10, wherein said hydrophilic extract of one or more herbal materials and said hydrophobic extract of said one or more herbal materials are the hydrophilic extract and hydrophobic extract of claim 1.

15. The nano-emulsion of claim 10 is prepared by:
a) extracting hydrophilic extract from herbs using a hydrophilic solvent, wherein said hydrophilic solvent is water;
b) extracting hydrophobic extract from herbs using a hydrophobic solvent, wherein said hydrophobic solvent is 95% ethanol;
c) dissolving said hydrophilic extract obtained in a) in an aqueous solvent to form an aqueous phase, and dissolving said hydrophobic extract obtained in b) in an organic solvent to form an organic phase;
d) mixing said aqueous phase and said organic phase with a surfactant to form a pre-mixture;
e) homogenizing said pre-mixture under 12,000-30,000 rpm for 10-30 mins to form the nano-emulsion, wherein said nano-emulsion is o/w type or w/o type nano-emulsion.

16. A transdermal patch comprises a backing layer, a matrix layer and a liner layer, wherein said matrix layer comprises the nano-emulsion of claim 10 and a adhesive material, the nano-emulsion is 1-25% by weight of the matrix layer and said adhesive material is selected from the group consisting of Carbomer 980, Carbopol U20, Carbopol HV-805EG, carboxymethylcellulose sodium, gelatin, poly(acrylic acid sodium salt), Poly(acrylic acid sodium salt) NP700, Polyvinylpyrrolidone 10, Polyvinvylpyrrolidone K90 and a combination thereof and said adhesive material is 10-15% by weight of the matrix layer.

17. The transdermal patch of claim 16, wherein said matrix further comprises at a crosslinking agent, a humectant, fillers, scaffold, a pH controlling agent or combination thereof.

18. The transdermal patch of claim 16, wherein 1-25% of the total mass of the matrix layer is the nano-emulsion, 10-15% is the adhesive material, 0.01-0.8% is the crosslinking agent, 15-25% is the humectant, 1-10% is the scaffold or filler and a pH controlling agent that maintains the matrix layer at a pH 5-6.

19. Use of the transdermal patch of claim 16 for treating traumatic injuries.

20. Use of the nano-emulsion of claim 14 for treating traumatic injuries.
